(19) United States
(12) Patent Application Publication
(10) Pub. No.: US 2012/0258197 A1
(43) Pub. Date: Oct. 11, 2012

(54) VINEYARD CULTURE METHOD ENABLING THE YEASTS THEREOF TO BE OBTAINED FOR HIGH SUGAR AND ALCOHOL CONTENT FERMENTATION

(76) Inventors: Miguel Jesús Marquez Sahuquillo, Venta Del Moro (Valencia) (ES); Juan Pedro Navarro Aviño, Valencia (ES)

(21) Appl. No.: 13/501,949

(22) PCT Filed: Oct. 15, 2010
(86) PCT No.: PCT/ES2010/000421

§ 371 Date: Jun. 28, 2012

(30) Foreign Application Priority Data
Oct. 15, 2009 (ES) P200901997

(51) Int. Cl. C12G 3/02; A01G 17/02; C12N 1/14; A21D 8/04

(52) U.S. Cl. 426/13; 426/18; 47/58.1 FV; 435/255.7

(57) ABSTRACT

Method for culturing resistant vineyards that selects indigenous vines, with natural stock, the fertilizer for which comprises local grasses/weeds, without other nutrients, harvesting being in the autumn. Fermentation method that is based on pressed grapes, in 20-25% of the vats, the wild yeasts thereof being produced and, in initial states, 3.5-6% vol. alcohol being achieved, with subsequent topping-up every 7-15 days; the total added sugar undergoes stepwise fermentation. Three different yeasts are obtained: white yeast (1), slow-growth yeast (2) and yellow yeast (3). Said yeasts make it possible directly to obtain high-quality beverages, such as cider, beers, cognacs, rums, vodkas, etc., with products having an alcohol content of up to 60% vol./vol. Fermentation also takes place with highly concentrated solutions of sucrose, molasses, sugars resulting from starch hydrolysis, and various plant materials. The production of bread, pastries and quality derived products is facilitated.

### OBJECT OF THE INVENTION

A first object of the invention relates to the development of a vineyard culture method resistant to meteorological and/or climatic roughness by developing deep and strong roots.

A second object of the invention relates to the development of a thorough fermentation method with wild grape yeasts reminiscing the soil and possessing its organoleptic properties as well as methods to obtain wines of high alcoholic grade.

A third object of the invention relates to the development of a method to obtain and use wild grape yeasts capable of fermenting sugars in low as well as high concentrations from sucrose solutions, commercial sugar, sugar cane molasses, beet molasses, fermentation of sugars from starch hydrolysis and fermentation of sugars from vegetal matter.

### BACKGROUND OF THE INVENTION

It is desirable for yeasts to complete fermentation of all sugar present in the grape juice during wine elaboration, thus a high alcohol degree, normally up to 14% in volume or not much higher, provides wine its final and aromatic structure.

Grape must fermentation in practice may be achieved through wild yeasts, inoculated yeasts or applying both procedures. Wild yeasts responsible for fermentation are naturally found in grape husks (generally as a fine white powder layer covering the grape's skin (vitis vinifera I.) named “pruina”. Specific cultured yeast strains yield specific fruitlike aromas, high alcohol degree, nose agreeable texture and other characteristics like fermenting at low temperatures or with relatively low pH, etc.

Most wine cellars normally use specific selected yeasts to turn sugar into alcohol; Saccharomyces cerevisiae being the best yeast species recommended for total alcohol fermentation.

Fermentation through wild or indigenous yeasts although not very extended, is mostly practiced by some European winemakers who traditionally ferment with wild yeasts with very good results. Likewise, Californian winemakers are also beginning to employ this spontaneous fermentation with very favorable results also. During vintage, grapes carry thousands of organisms, yeasts included, with the ensuing risk of infection. One of the most common characteristics of wild yeasts resides in its low resistance to alcohol and scarce resistance to infections.

However, many types of wild yeasts are incapable of acting once a 9% vol. alcohol level is reached, clogging fermentation and resulting in a wine without consistency, with a low immunity system and a great amount of residual unwanted sugar among other problems. Time needed for wild yeasts to establish colonies, wherein must remains exposed to infection by other organisms having a faster development or to oxidation, is an additional difficulty encountered when using wild yeasts in fermentation; besides, once fermentation begins, it is a lengthy and slow process with no guarantee of a satisfactory ending. The unpredictability of aromas and esters introduced by wild yeasts in wine is also a problem; however, selected or commercial laboratory yeasts enable attainment of such flavors and aromas as desired by winemakers' taste.

The fact that it takes longer to initiate fermentation, allowing more contact time with the grape's skin, thus translated into more body, depth of character, color and more fruitlike flavors (that is, much more variables than with industrial wines) is an advantage related to employment of wild yeasts. There is confirmation that many of the unpredictable aromas and esters conveyed by wild yeasts confer an interesting sophisticated nature to wine, resulting in a wine with complex flavors, good bouquet and very good alcohol.

A way to influence vintage is through irrigation; however, the effect of irrigation in relation to the wines' alcoholic grade redounds in a decrease in concentration of sugars due to the effect of dilution. The production increases when comparing irrigated and non irrigated grapevines, observing that phenol compounds and intensity of colorants decrease in wines from irrigated grapevines. The must shows an increase in malic acid in grapes from irrigated grapevines. The effect of water contributions at the end of maturation influences negatively in the composition and quality of final wine due to a decrease in concentration of elements, owing to dilution produced by water in the berry.

In known art there are very few works exploring in depth factors influencing wine quality, since factors influencing the fermentative glycolysis of sugars by wild yeasts are complex, due to existing interrelation among them and to the nature of parameters intervening during the fermentation process.

Object of paper FR 2.844.275 relates to a natural wine fermentation procedure with wild yeasts characterized by the following steps:
transferring grapes to wooden casks through gravity,—using a vibrating motion to insure full filling of casks,
grape fermentation control in these casks with a periodic oscillation, etc.,
further malo-lactic wine fermentation and conservation of this wine in the same fermentation cask.

It does not employ pumps or tanks for reshuffling, only barrels.

Patent WO 2004.029.193 presents a production method for a fermentation product comprising a fermentation stage including contact with a microorganism in fermentation or fermentation means used with at least an esterase enzyme as for example: lipase, phitase, phospholipase and cutinase.

However, in known art there are no references to vineyard culture focusing on attainment of wild yeast strains to perform total sugar fermentation.

A method to obtain wild yeasts achieving total sugar fermentation and guaranteed to overcome wine infections would undoubtedly be of practical interest.

### DESCRIPTION OF THE INVENTION

The present invention is related to a culture method of a vineyard resistant to meteorological and/or climatic roughness through development of deep and strong roots, a culture method through which wild yeasts achieving the wine fermentation process develop; and a method of thorough fermentation to obtain total transformation of sugar into alcohol, developing and multiplying these wild yeasts in grape juices and achieving a high alcohol degree, totally superior to that obtained through conventional fermentation practiced to date.

The natural culture method consists in obtaining a totally natural wine, starting by grapevine selection, such that the grapevine develops and produces fruit by itself and through its own yeasts. Human intervention focuses on the preparation of the soil, cultivating the herbs for further burying as organic yield to the soil “in situ”, with no addition of fertilizer whatsoever, the plant becoming strong in a natural way and adapting to the soil by itself. Following this procedure there is confirmation through the years that harvest could be delayed, thus obtaining grapes with more sugar proportion; collecting even in January and February, although in recent years it has been possible to advance it somewhat, the result remaining the same as to quality and production increase.

The method begins from the very moment of vine selection until yeast is obtained from these strains, to be genetically treated further in their case, achieving a faster multiplication and to be predominant in the fermentation process, that is, developing in such quantity and strength as to prevail in face of other yeasts that might appear, furthermore attaining total fermentation of sugars to obtain a wine with character and very good alcohol, rendering it longevity, aromas and flavors, that are not only reminiscences of the fruit but also of the soil and environment where the grapevines have been cultivated at, solving an actual problem consisting in that fermentation with a high degree of alcohol could not be obtained through wild or indigenous yeasts.

Essential characteristic stages of the natural culture method are:
selection of a variety of natural autochthonous grapevines, having a natural origin with no grafting,
fertilizing said grapevines with compost obtained from the soil's own organic matter,
collection of grapes in a delayed fashion,
no addition of other nutrients than those of the soil itself.

The thorough fermentation method with wild grapes' yeasts reminisce the soil possessing his organoleptic properties; and more important, they ferment the totality of sugars contained in the grapes. The thorough fermentation method does not employ any type of additional yeast, fermenting only through wild yeasts of the own grapes, that is, fermentation is accomplished with yeasts obtained from grapes grown by the natural culture method. These wild yeasts of the own grapes whose properties greatly reminisce the soil are capable of fermentation even with high alcohol percentages. In wine cellars there is evidence of an evolution in time of wild yeasts yielded by grapes, wherein an enhancement and standardization of flavors, aromas and bouquet has taken place, as alcohol was ever much better until 2004, wherein its properties have stabilized, yielding since then wines more regular as to their properties.

Furthermore, the thorough fermentation method with wild grapes' yeasts enables obtainment of grape juices with ever more superior alcohol contents, allowing direct obtainment of alcoholic beverages as cognacs, etc. Finally, the method of the present invention creates conditions for industrial production of bio-ethanol with more effective energetic ratings, in comparison to those presently obtained in industrial production of bio-ethanol through fermentation of raw materials such as sugar cane or beet juice.

Essential characteristic stages of the thorough fermentation method are:
formation of a barrel wild grapes' juice with pressed grapes,
fermentation in vats of less than 5,000 liters capacity over 20-25% of said volume,
reproduction of obtained wild autochthonous yeasts, achieving 3.5 to 6% in volume of alcohol,
further topping ups every 7 to 15 days,
stepwise fermentation with the same yeasts, achieving fermentation in successive stages, obtaining in each stage an increase in of alcohol percentage, until fermentation of all the sugar and obtaining a wine of final alcoholic grade between 14 to 21% in volume.

### DESCRIPTION OF THE DRAWINGS

FIG. 1 corresponds to sediment of a cask containing 17.7% ethanol and sediment of a second cask with 18.2% alcohol content, both seeded in plates in YPD medium (yeast extract-Peptone-Dextrose).

FIG. 2 shows in a seeding in YPD medium White Yeast 1, Slow Growth Yeast 2 and Yellow Yeast 3.

FIG. 3 presents a sample of the lower part of the tank and another of the grape fermentation tank sediment of the 2008 vintage, in YPD medium, appreciating filamentous fungi that might belong to slow growth yeast.

FIG. 4 equally corresponds to the 2008 vintage, presenting a sample of the lower part of the tank and another of the grape fermentation sediment, in YPD medium, appreciating filamentous fungi, abundant presence of white yeast and a small presence of yellow yeast.

In FIGS. 5.1 and 5.2 the right hand column is an YPD medium without glucose undergoing an increase in its ethanol level. The left hand column shows plates of YPD medium containing 1% glucose. At the left hand side of the photos, the text indicates ethanol percentage in each plate. All plates are divided into three zones seeding each respective zone with each of the White Yeast 1, Slow Growth Yeast 2 and Yellow Yeast 3 strains.

FIG. 6 presents behavior of the three yeast strains White Yeast1, Slow Growth Yeast 2 and Yellow Yeast 3 in YPD medium with 1% glucose at elevated ethanol levels. Equally, all the plates are divided into three zones seeding in each zone each one of the strains.

FIG. 7 presents microscopic view morphology of separated white yeast seeding.

FIG. 8 presents microscopic view morphology of separated white yeast seeding after an advanced growth.

FIG. 9 corresponds to microscopic view of separated slow growth yeast seeding.

FIG. 10 corresponds to the same FIG. 4 plate from which some hyphas—cylindrical filamentous elements characteristic of the majority of fungi-, are obtained in a fermentation must are to be analyzed.

FIG. 11 presents one of the hyphas whole, including its head.

FIG. 12 presents an enlargement of the hypha's head of FIG. 11, observing it is made up of small cells.

FIG. 13 is an enlargement wherein small cells of FIG. 12 are observed.

FIG. 14 is a photograph of a foamed flask, said foam due to CO₂ emission from the sample in fermentation in a medium with 60% commercial sugar (sucrose).

FIG. 15 is a photograph of a foamed flask, said foam due to CO₂ emission from the sample in fermentation in a medium with 70% commercial sugar (sucrose).

FIG. 16 is a photograph of a foamed flask, said foam due to CO₂ emission from the sample in fermentation of beet molasses with 12.2% sucrose content.

FIG. 17 is a photograph of a foamed flask, said foam due to CO₂ emission from the sample in fermentation of pure sugar cane molasses with 40% sucrose content.

FIG. 18 is a photograph of a foamed flask, said foam due to CO₂ emission from the sample in fermentation of beet molasses with 12.2% sucrose content.

FIG. 19 is a photograph of a foamed flask, said foam due to CO₂ emission from the sample in fermentation of beet molasses with 18.4% sucrose content.

FIG. 20 is a photograph of a foamed flask, said foam due to CO₂ emission from the sample in fermentation of beet molasses with 42.7% sucrose content.

FIG. 21 is a photograph of a foamed flask, said foam due to CO₂ emission from the sample in fermentation of sugars from starch with 20% starch concentration.

FIG. 22 is a photograph of a foamed flask, said foam due to CO₂ emission from the sample in fermentation of sugars from starch with 40% starch concentration.

FIG. 23 is a photograph of a foamed tube, said foam due to CO₂ emission from the sample in fermentation of sugars from vegetal matter.

FIG. 24 is a photograph of a foamed tube, said foam due to CO₂ emission from the sample in fermentation of sugars from vegetal matter.

### PREFERRED EMBODIMENTS

### Embodiment No. 1. Resistant Vineyard Culture Method

a). Culture Conditions:

Work initiated in 1989, selecting Bobal, Crujidera, Royal and Tardana autochthonous grapevines. Since the very beginning agriculture was totally natural, no chemical or organic fertilizer was employed except compost elaborated with herbs from the soil wherein the grapevines were planted. The former object corresponds to the First Object of the Invention, resistant vineyard culture method, consisting in achievement of resistant grapevines by themselves, able to be self-sufficient and relying on the soil's own nutrients, thereby developing deep and strong roots. Thus, the grape acquires flavors and aromas reminiscing the soil they are cultivated at. There was no irrigation though rainfall rate in the area was under 500 l/m², typical of a continental dry climate (between 300 to 500 mm rainfall). This also corresponds to the First Object of the Invention for the culture type, furthermore enabling adaptation to this climatic condition of yeasts present in the grapes' skin.

b) Collection Chronology and Characteristics of Grapes:
Year 1990: grape was collected later on, since grapes held on perfectly due mainly to culture manner. These grapevines extended across the land, the plantation having a south orientation and located in a microclimate, since there were two small woods at both sides of same.
Years 1996 and 1998: grapevines started production of grapes of different flavors and aromas after 6, 7 years, acquiring a better production quality every year. Wine was made with lately collected grapes, corresponding to the Second Object of the Invention. This type of late culture attains a high alcoholic grade between 14° and 16° alcohol in a totally natural manner, relying only on wild yeasts appearing in the must that is, greatly surpassing the generalized opinion that wild yeasts hardly survive and develop in media with such high alcohol contents. Although they do not acclimated to more alcohol graduation, by 1998 they changed aromas. It is known that yeasts are very sensitive living beings and it seems they are gradually acclimating to these high alcohol graduations.
Year 2002: According to the First Object of the Invention, the first commercial harvest was collected through a late vintage, in times of fog and especially after autumn rainfall. According to the invention, although normally when it rains in September grapes fatten; nevertheless, autumn rainfall thickens the skin of grapes impeding their fattening, thus appearing here the yeasts of the Second Object of the Invention: wild yeasts thorough fermentation method, said yeasts intervening further in the innovative sugar fermentation process.

### Embodiment No. 2. Wild Yeasts thorough Fermentation Method

According to the Second Object of the Invention, wine is obtained:
elaborating a wild grapes' barrel with pressed grapes, in vats of less than 5,000 liters, over 20-25% of said volume, or an equivalent proportional relation, to reproduce the yeast, that is, the wild or natural yeast of the grapevine.
fermentation duration: 15 to 20 days reaching 3.5 to 6% volume of alcohol.
from this wild grapes' barrel, 15 to 20% of the container's volume was added every 7 to 15 days, performing a stepwise fermentation and contributing in this manner oxygen and nutrient to the yeasts, raising alcoholic graduation until all the sugar is fermented, and
achieving final alcoholic grade between 16-19% alcohol volume.

2.1 Production since 2002: In 2002 and 2003 juices were produced with 15.5° alcohol, obtaining in 2004 a concentration of alcohol between 15.5 and 18.98% volume.

In this manner a wine is obtained with body and very good alcohol, thus assuring its quality in time and with very good flavors, reminiscing the environment and the soil, said aromas reminiscing the fruit they came from and obtaining a characteristic bouquet and personality that, due to the longevity of the grapevines (old vineyards with more than 70 years), obtaining a greater quality in fermented juices.

2.2 Characterization of Autochthonous Wild Yeasts

According to the Third Object of the Invention, Method to obtain autochthonous wild yeasts, yeasts causing fermentation corresponding to the Second Object of the Invention were subjected to laboratory studies, extracting representative samples of the wine produced.

Samples: Collected samples belonged to the 2004 vintage and were:
sample of sediment of a cask containing an ethanol percentage of 17% alcohol volume, 17.7% from now on.
sample of sediment of another cask which alcohol content was 18.2% alcohol volume, 18.2% from now on.

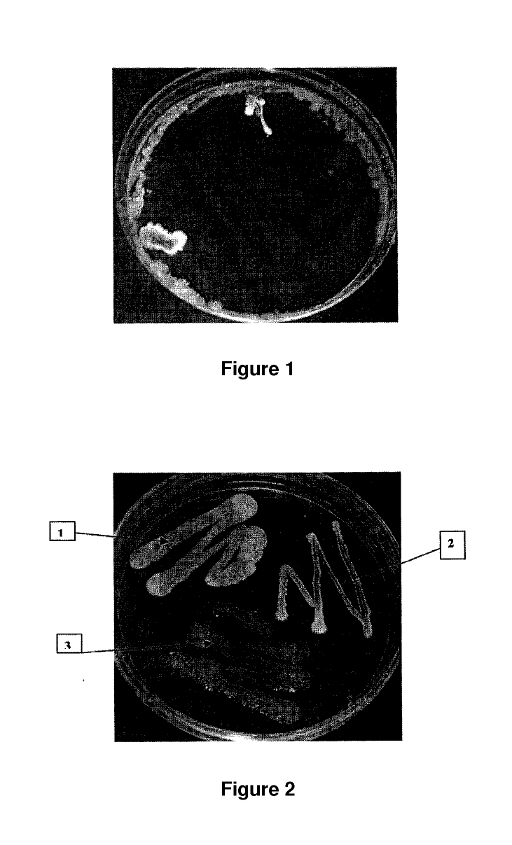

Study of samples in plates:
1—Samples were seeded in plates containing YPD. FIG. 1 shows an example of the results obtained wherein three types of different colonies were observed.
2—A sample of the three types of yeast was taken and seeded in a new plate in YPD medium so they could grow in greater amount and could be better differentiated, see FIG. 2. An arrow points out each one of the yeasts in this FIG. 2, thus distinguishing White Yeast 1, Slow Growth Yeast and Yellow Yeast 3.
3—With the yeasts perfectly defined and differentiated, its growth capability was determined. The same amount of each type of yeast was taken and seeded in a new YPD plate confirming the so called slow growth yeast in fact grew at a slower rate than the other two.
4—New samples were taken, not from casks but now from the fermentation tank and the 2008 vintage.
a sample from the liquid in the bottom part of the tank was taken.
another sample of the sediment in the fermentation tank was taken.

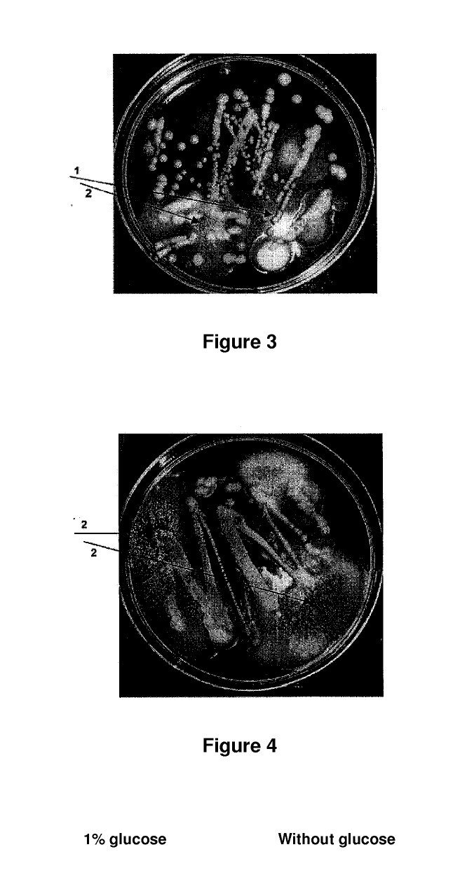

The two samples were seeded in two plates in YPD medium observing occurrence of some filamentous fungi indicated in FIG. 3 and better viewed in FIG. 4 with an arrow, supposedly coming from slow growth yeast 2 that, in the fermentation process is found in form of filamentous fungi, to further evolve to the yeast form obtained in casks. In the plates there was also abundant white yeast 1 and in a lesser amount yellow yeast 3.

2.3 Tests of Isolated Yeasts at Different Ethanol Concentrations

a.—Seeding of glycerinates from the three yeasts—Growth of yeasts at different ethanol and glucose concentrations.

Glycerinates of each of the three yeasts were carried out and seeded in media with different ethanol levels to see their development capability. Two types of media were prepared, one with glucose (1%) and another one without glucose. After some unsuccessful preparation intents in YPD medium with ethanol since elevated concentrations of ethanol fluidize in excess the YPD medium, an adequate procedure was carried out. The medium solidified by increasing the amount of agar depending on the amount of ethanol to be further added. The yeasts were seeded in these plates, carefully seeding the same amount of yeasts in all plates, obtaining the following results, see FIGS. 5.1 and 5.2:

1.—White yeast grows appropriately in all ethanol concentrations and in great amounts either in medium with glucose (1%) or in medium without glucose, for it can use as feeding source the carbon of ethanol as well as the peptone and the yeast extract, all of them components of the YPD medium.

2.—Yellow yeast presents a different morphology depending on the medium containing glucose or not. In glucose media it grows in big colonies, while in media without glucose it grows in smaller colonies, but in a much greater number of said colonies; producing in both cases a reduction of the quantity of yeast obtained by increasing the percentage of ethanol.

3.—Slow growth yeast as well as yellow yeast grows in a different manner depending on the medium containing glucose or not. In this case, the behavior is opposite to that of yellow yeast, thus colonies in medium without glucose present a bigger size than colonies in medium with glucose, although in smaller number of colonies. As to growth in different ethanol levels, at higher ethanol levels they grow with more difficulty.

b.—Yeasts growth at higher ethanol concentrations

In further tests ethanol concentration was increased to determine ethanol limit concentration resisted by each type of yeast. This test posed a problem since the amount of ethanol to be added was rather high and the medium could have problems in solidifying, so the decision was to increase concentration of agar in the medium from 2% to 3%. The amount of glucose employed in the medium was 1%.

The following results were obtained:
White yeast grew significantly in all ethanol concentrations. The difference between concentrations was the yeast growth rate. For higher concentrations, growth rate is smaller, thus it took the yeast more days to grow but as can be observed, the amount of grown yeast is very similar in all plates.
Yellow yeast grew in all plates, but its growth was small, in some plates it is difficult to observe if it has grown at all. Therefore, greater growth was observed in plates with 25 and 40% ethanol volume.
Slow growth yeast grew in a great number of colonies for a concentration of 25% volume and for higher ethanol concentrations it did not experiment any growth whatsoever.

Therefore in a preliminary way as a conclusion, slow growth yeast does not resist ethanol concentration conditions over 25% volume, while white yeast resists very high concentrations of ethanol; as for yellow yeast it is very difficult to grow in all ethanol concentrations, thus growing in very small amounts.

c.—Yeast tests at even higher ethanol levels

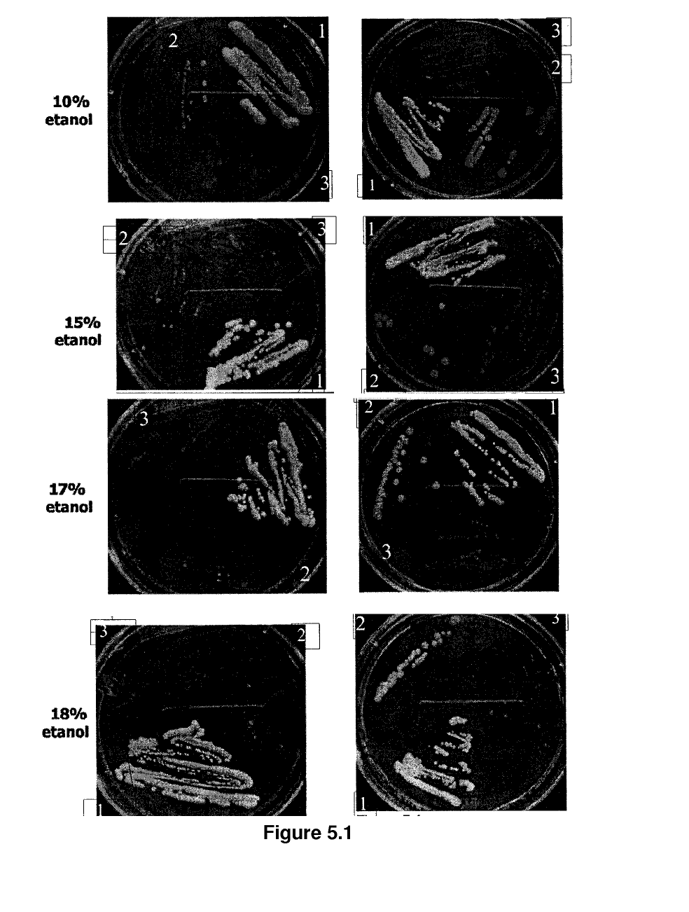

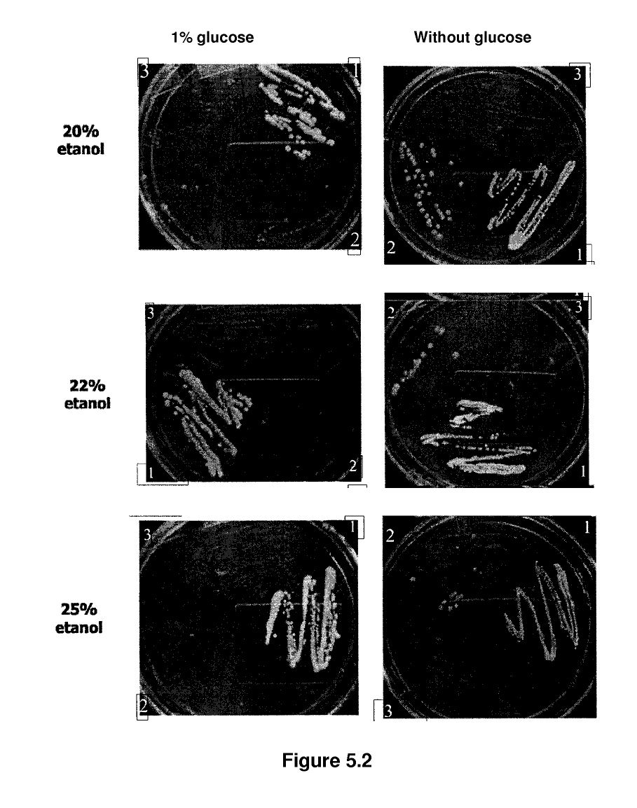

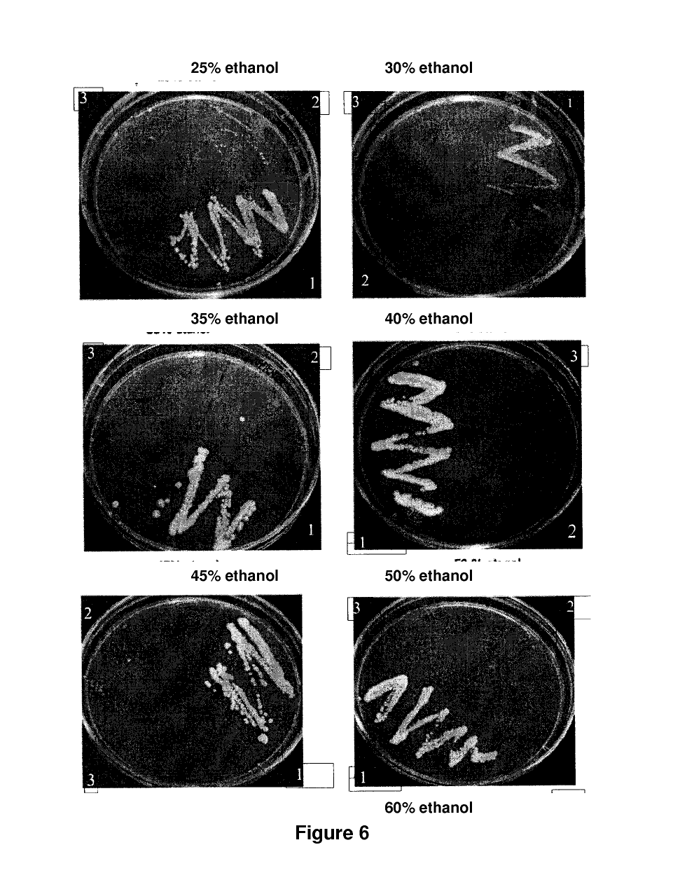

Tested ethanol levels in these plates were: 40%, 45%, 50% and 60% volume. Results obtained (see FIG. 6), were:
White yeast grows significantly in all plates, although at 60% ethanol volume there is a marked decrease in the amount of yeast, possibly because at this level it is affected by toxicity of ethanol.
Slow growth yeast doesn't grows in any plate.
Yellow yeast does present signs of growth in some of these plates but it seems to be a residual growth due to the great amount of yeast seeded with the handle and remaining in the seeding line.

d.—Morphology of the different yeast types

The different types of yeasts were observed under the microscope.

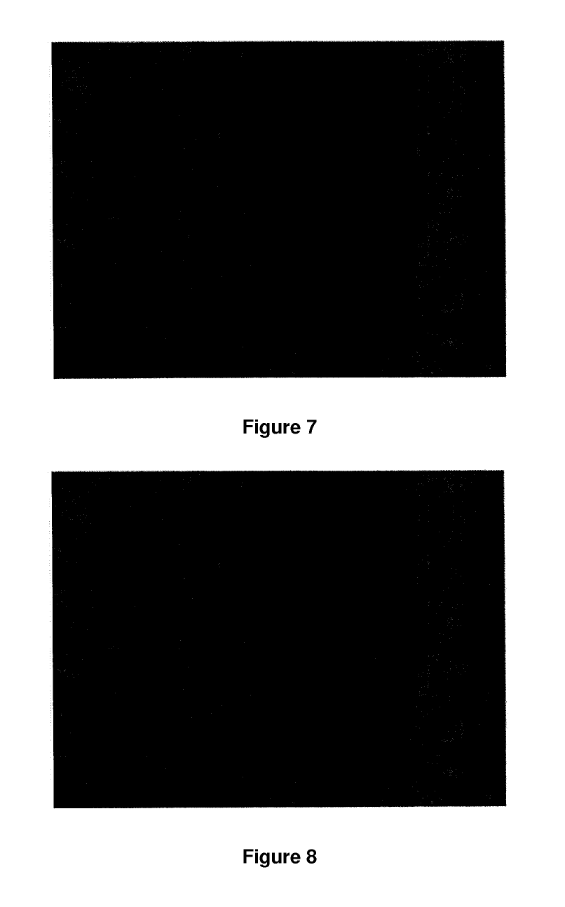

FIGS. 7 and 8 show images corresponding to white yeast 1. FIG. 8 shows a great number of cells in reproduction.

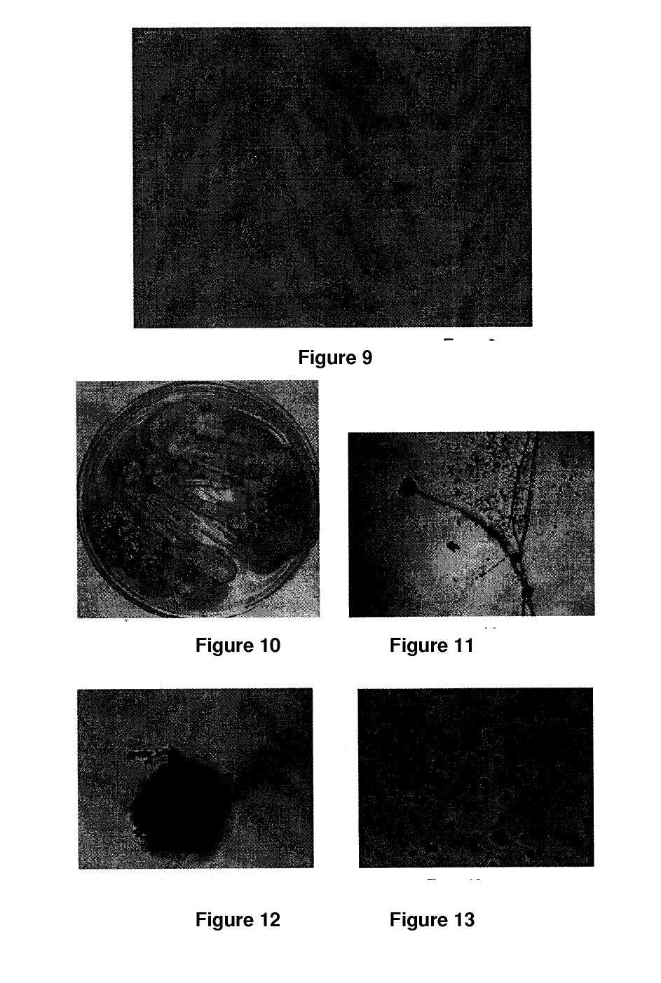

FIG. 9 shows images corresponding to slow growth yeast 2.

FIG. 10 shows images corresponding to must in fermentation collected from the fermentation tank of the 2008 vintage, wherein hyphas are appreciated.

FIG. 11 shows images corresponding to a whole hypha of FIG. 10, perfectly observing said hypha's head.

FIG. 12 shows images corresponding to an enlargement of the hypha's head of FIG. 11, observing it is formed by small cells.

FIG. 13 shows images corresponding to small cells forming the hypha's head of FIG. 12. These cells resemble those observed in the slow growth yeast 2, see FIG. 9; although the size of the hypha's head cells is bigger and its cellular membrane thicker than those of the cells observed in the slow growth yeast 2.

### Embodiment No. 3. Capability of Mixed Cells in Sucrose (Commercial Sugar) Fermentation

A culture medium is prepared in flasks with different concentrations weight/volume of commercial sugar. The medium components are:
monopotassic phosphate 0.5%
ammonium sulphate 0.2%
heptahydrated magnesium sulphate 0.004%
yeast extract 0.1%
water

Sugar concentrations are: 1, 2, 10, 15, 20, 25, 30, 40 and 50%, all of them in weight/volume percentage.

Preparation of white yeast 1, yellow yeast 3, slow growth yeast 2 strains to be inoculated in different media:

The yeasts are seeded in liquid medium YPD (5 ml), colonies of the different strains grown in plates of YPD medium, each strain separately, and agitated during 24 hours at 240 rpm and 28° C. Once this time has elapsed and all the strains grown, 20 μl of each of them are placed in a test tube with 5 ml of the corresponding medium in each tube, agitating them at 240 rpm during 24 hours.

Once this time has elapsed each medium presents the following growth

| | Test | | | | | | | | |
|---|---|---|---|---|---|---|---|---|---|
| | No. 1 | No. 2 | No. 3 | No. 4 | No. 5 | No. 6 | No. 7 | No. 8 | No. 9 |
| Conc_{sugar} | 1% | 2% | 10% | 15% | 20% | 25% | 30% | 40% | 50% |
| OD₆₀₀ | 5.74 | 7.48 | 8.35 | 6.97 | 6.21 | 5.21 | 5.51 | 3.84 | 4.40 |

Conc_{sugar}: sugar concentration.
OD₆₀₀: optical density at 600 nm.

Absorbance is measured in the tests to evaluate cellular growth in culture medium. Measurement of absorbance is directly related to the amount of yeast cells in a determined volume of culture medium. The difference measured by spectrophotometer between intensity of light emitted by the lamp and that reaching the detector once the sample is traversed, that is, the amount of light absorbed by the cells should be greater corresponding to a greater number of cells in the sample.

These growth values afford an idea that these yeasts have great resistance to high sugar concentrations.

The test tube content was transferred into a flask with culture medium at its corresponding concentration, leaving a total volume of 40 ml. It was agitated at 240 rpm during 4 hours, so inoculated cells would multiply. Once this time elapsed the agitation stopped leaving cultures at 31° C. in anaerobic conditions. A small agitation (22 rpm) followed so cells would be well spread in the whole culture. At this time the fermentation begun. The carbohydrates' concentration was determined the following week by the Phenol-Sulphuric Dubois Colorimetric Method obtaining the following percentages:

| Initial sugar | Final sugar |
|---|---|
| percentage (%) | percentage (%) |
| 1 | 0.052 |
| 2 | 0.049 |
| 10 | 0.947 |
| 15 | 4.988 |
| 20 | 9.506 |
| 25 | 13.024 |
| 30 | 21.988 |
| 40 | 31.137 |
| 50 | 40.333 |

Therefore reduction of sugar contents is observed in all the flasks, indicating fermentation has been accomplished at all tested concentrations.

The amount of ethanol that could be found in these cases is theoretically described as follows: for example, for the initial 20% sugar concentration a final concentration of 9.5% sugar is obtained, this means that 10.5 g. of sugar are consumed in 100 ml of initial juice.

The reaction describing the alcoholic fermentation is as follows:

C₅H₁₂O₆+2Pᵢ+2 ADP→2CH₃—CH₂OH+2CO₂+2 ATP

The stoichiometric relation expresses that by each mol of glucose two mol of ethanol are obtained.

100 ml of juice→10.5 grams glucose

10.5 grams glucose→0.058 mol. glucose (MP glucose=180.16 gr/mol)

0.058 mol glucose→0.116 mol ethanol (stechiometry 1:2)

0.116 mol ethanol→5.370 grams ethanol (MP ethanol=46.07 gr/mol)

5.370 grams ethanol→6.806 ml ethanol (ethanol density=0.789 gr/ml)

Summary: 100 ml of juice→6.806 ml ethanol. Considering real yield is 80% of the theoretical: 6.806×0.8=5.445 ml of ethanol. That is, in the example from an initial 20% concentration of sugar a final approximate concentration of alcohol of 5.4% is obtained.

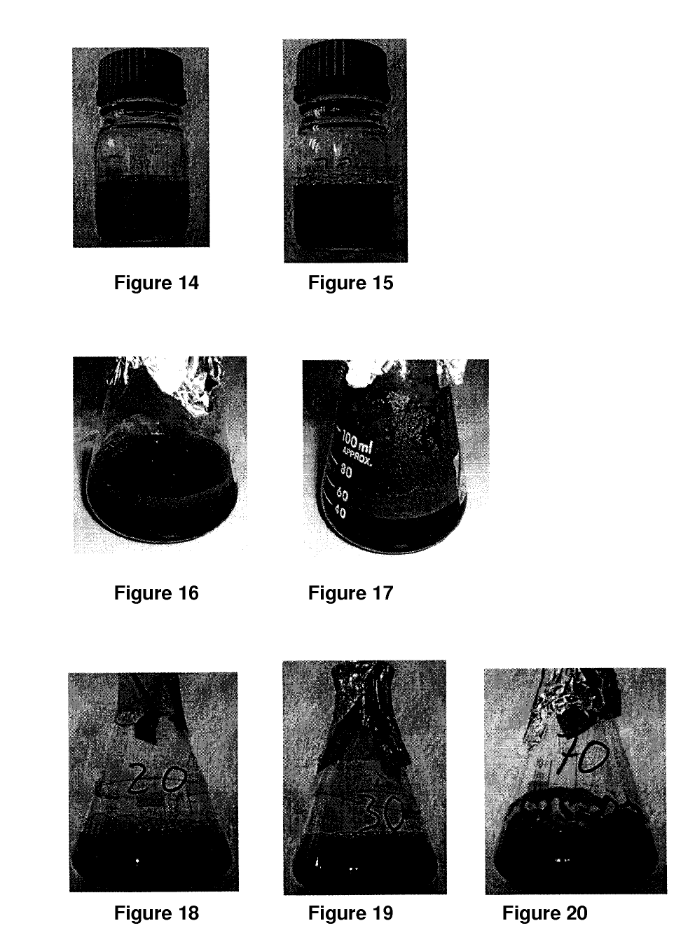

Since it seems these yeast strains resist without difficulty sugar concentrations up to 50%, as following step the sugar concentration was increased. Tests were made with 60 and 70% (weight/volume) sugar concentrations, see FIGS. 14 and 15, observing the formation of foam produced by CO₂ bubbles from fermentation. These media were agitated during 72 hours at 28° C. and 240 rpm. To check that strains grow with no difficulty in these media so concentrated in sugar, the optical density of these two cultures was measured and the results obtained were:

| % Sugar | 60 | 70 |
|---|---|---|
| OD₆₀₀ | 12.6 | 6.1 |

It is confirmed that strains grow even in medium with a 70% sugar concentration; at the end of fermentation of this juice theoretical alcoholic grade to be obtained would be 45.37°. Calculation algorithm of this datum is presented further.

The reaction describing alcoholic fermentation is as follows:

C₆H₁₂O₆+2 Pᵢ+2 ADP→2CH₃—CH₂OH+2CO₂+2 ATP

The estechiometric relation shows that for each mol of glucose two mol of ethanol are obtained.

100 ml of juice→70 grams glucose

70 grams glucose→0.338 mol glucose (M.B. glucose=180.16 gr/mol)

0.338 mol glucose→0.777 mol ethanol (stoichiometric 1:2)

0.777 mol ethanol→35.8 grams ethanol (MP ethanol=46.07 g/mol)

35.8 grams ethanol→45.37 ml ethanol (ethanol density=0.789 g/ml)

Summary: 100 ml of juice→45.37 ml ethanol. Considering real yield is 80% of the theoretical: 45.37×0.8=36.30 ml of ethanol, or a concentration of 36.35 vol.

### Embodiment No. 4. Capability of Mixed Strains in Molasses Fermentation (Obtained from Sugar Cane) with Different Dilutions

Sugar cane molasses were used as base for culture medium. From molasses different dilutions were made to test behavior of strains before different sugar levels. The starting molasses had 78.9° Brix and 49.9% sucrose content.

The following table shows prepared media:

| | Composition of the mixtures water/molasses. C_{H2O/Me} | | | | | |
|---|---|---|---|---|---|---|
| | No. 1 | No. 2 | No. 3 | No. 4 | No. 5 | No. 6 |
| Water | 150 ml | 120 ml | 100 ml | 120 ml | 40 ml | |
| Molasses | 50 ml | 60 ml | 90 ml | 180 ml | 160 ml | pure |
| | | | | | | molasses |
| % sucrose | 12.5% | 16% | 23% | 30% | 40% | 50% |

Procedure for strain inoculation is as follows:

One colony of each strain, White Yeast 1, Slow Growth Yeast 2 and Yellow Yeast 3 is seeded in YPD medium re-suspending them together in 1 ml of water. A 5 ml aliquot of each prepared medium is placed in test tubes, inoculating 50 μl of the strains' mixture in each tube. It is agitated at 240 rpm and 28° C. At 66 hours the strains show the following growth.

| | No. 1 | No. 2 | No. 3 | No. 4 | No. 5 | No. 6 |
|---|---|---|---|---|---|---|
| %_{sugars} | 12.5 | 16 | 23 | 30 | 40 | 50 |
| OD₆₀₀ | 2.66 | 0.5 | 0.09 | 0.34 | 0.44 | 0.00 |

OD₆₀₀ values of each sample are independent from the others, it being proof of how yeasts have adapted to that medium.

The following step consists in transferring an aliquot of a 1/1000 dilution of the content of these tubes to its corresponding flask. Once the yeasts are inoculated to the flask, agitation is applied at 240 rpm and 28° C. At 16 hours they showed the following growth:

| | No. 1 | No. 2 | No. 3 | No. 4 | No. 5 | No. 6 |
|---|---|---|---|---|---|---|
| %_{sugars} | 12.5 | 16 | 23 | 30 | 40 | 50 |
| OD₆₀₀ | 2.48 | 1.05 | 0.33 | 0.33 | 1.01 | 0.00 |

From 16 hours on the agitation is reduced to decrease oxygen supply allowing beginning of fermentation. FIGS. 16 and 17 show formation of foams produced by the presence of liberated CO₂, as product of the fermentation. FIG. 16 corresponds to a flask in which the sucrose concentration is 23%. FIG. 17 corresponds to a flask in which the sucrose concentration is 40%. It should be noted that molasses is a very viscous fluid thus cells find much resistance to spread through same, that is the reason why when molasses is not diluted with some water, cellular growth results practically impossible as can be seen in case No. 6, resulting in pure molasses. Thus, strains grow in 40% sucrose with no difficulty; at the end of fermentation of this juice the theoretical alcoholic grade to be obtained would be 25.93° and according to calculus, yielding an equivalent 20.7% volume.

Another experiment was carried out with molasses from beets; these molasses had a 76.8° Brix concentration. Sucrose content of molasses was 47%. The following molasses dilutions were prepared for the preparation of media:

| % sucrose | Components |
|---|---|
| 6.1 | 43.49 ml water + 6.51 g. molasses |
| 12.2 | 37 ml water + 13 g. molasses |
| 18.4 | 30.47 ml water + 19.53 g. molasses |
| 24.4 | 23.96 ml water + 26.04 g. molasses |
| 27.5 | 20.7 ml water + 29.3 g. molasses |
| 30.5 | 17.45 ml water + 32.55 g. molasses |
| 36.6 | 11 ml water + 39 g. molasses |
| 42.7 | 4.42 ml water + 45.58 g. molasses |
| 47 | 50 g. molasses |

In these media studied strains of White Yeast 1, Slow Growth Yeast 2 and Yellow Yeast 3 were inoculated and agitated at 240 rpm and 28° C. Observed growth after a week of agitation was:

| % sucrose | OD₆₀₀ |
|---|---|
| 6.1 | 12.88 |
| 12.2 | 11.22 |
| 18.4 | 14.3 |
| 24.4 | 13.32 |
| 27.5 | 12.36 |
| 30.5 | 9.4 |
| 36.6 | 2.54 |
| 42.7 | 2.34 |
| 47 | — |

This table indicates how from 30.5% sucrose, yeasts find more resistance for reproduction, manifesting in a decrease in absorbance, although still at 42.7% persists formation of foam having enough capability to carry out alcoholic fermentation.

This can be seen in FIGS. 18, 19 y 20. FIG. 18 shows a flask with molasses in which sucrose concentration is 42.7%; FIG. 19 shows a flask with molasses in which sucrose concentration is 18.4%, FIG. 20 shows a flask with molasses in which sucrose concentration is 12.2%. Results obtained, see FIG. 18, demonstrate that a juice can be fermented with sugar content up to 42.7%, this presuming a 27.68%, 28% volume/volume ethanol (see theoretical calculus method previously presented herewith), something really extraordinary. As pointed out since molasses is a very viscous fluid, cells find much resistance to spread through it, so when molasses is not diluted in some water, cellular growth is practically impossible.

### Embodiment No. 5. Capability of Mixed Cells in Sugars' Fermentation from Starch Hydrolisis

The present embodiment is unquestionably of practical interest since starch is the origin of treatments for production of both, bread or beer. Different dissolutions of starch in water were prepared; starch is not soluble in water so it remains suspended. To hydrolyze it a method of acid hydrolysis was used consisting in lowering pH to 0.8 with sulphuric acid and agitation at 84° C. during 6 hours.

Starch concentrations used were: 10%, 20%, 40% and 50%, all of them referred as weight/volume percentage. Once the starch was hydrolyzed pH was neutralized with NaOH and strains from a saturated culture in YPD medium inoculated; a saturated culture is one in which the number of cells remains constant, that is cells die and are born in the same number. 170 μl of culture from each strain, the mixture of White Yeast 1, Slow Growth Yeast 2, and Yellow Yeast 3, were inoculated.

At 48 hours of agitation at 240 rpm and 28° C. the media presented the following growth:

Initial percentage of starch in the flask

| % | OD₆₀₀ |
|---|---|
| 10 | 0.749 |
| 20 | 0.556 |
| 40 | 0.471 |
| 50 | 0.351 |

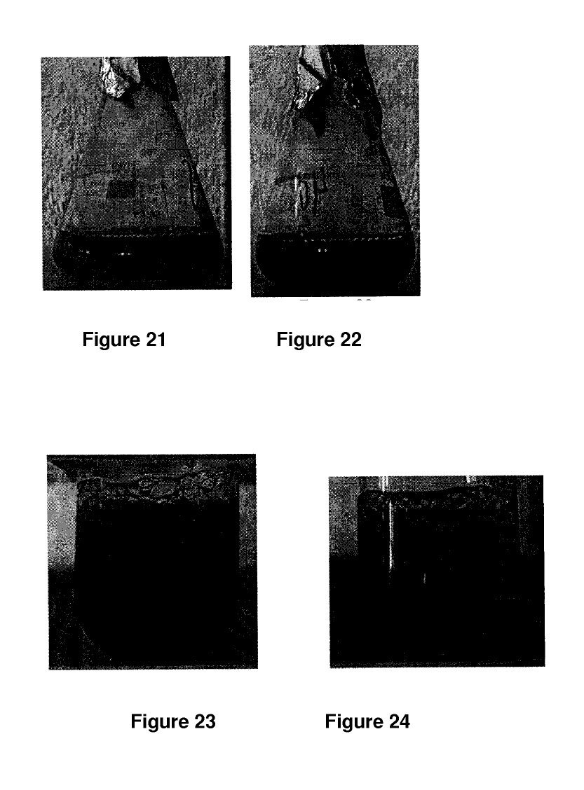

As shown in the previous table, the lower OD₆₀₀ correspond to flasks prepared with a higher starch concentration wherein yeasts find higher resistance to survive and thus their number is smaller. At 48 hours agitation stops so there is no oxygen supply, allowing end of fermentation. FIG. 21 corresponds to a flask prepared with 20% starch. FIG. 22 corresponds to a flask prepared with 40% starch.

Since flour contains starch and yeasts are capable of fermenting starch, the use of these three yeast strains simultaneously in the fermentation process may confer higher quality, whatever the flour fermentation product desired, since the coordinated action of the three yeasts will enhance in itself the fermentation process. Yeast that works better in glucose (Yellow yeast 3) can cover the other two in media with a high content of same (they must survive in 70% sucrose anyhow), Low Growth Yeast 2 can create a veil favoring oxygen absence, providing all the advantages to produce a better fermentation and lastly, White Yeast 1 can grow in medium of high ethanol concentrations and maybe protect the other yeast species present in the process. Consequently in any process where fermentation from fermentable sugars is present, these yeasts can presumably provide a higher quality to the product obtained.

### Embodiment No. 6: Capability of Mixed Strains from Vegetal Matter in Sugars' Fermentation

To carry out this experiment the Nicotiana glauca species was selected as an example of vegetal matter from wild plants, taking samples from different parts of the plants as root, green stalk, woody stalk and leaves. To obtain the plants' sugars, these were crushed and the crushing mixed with water, so soluble sugars of the plants dissolved in water. At this point, the amounts (in percentage weight/volume) of sugar present in the media were:

| | Initial sugar |
|---|---|
| Sample | percentage (%) |
| green stalk | 1.98 |
| woody stalk | 1.53 |
| leaf | 2.19 |
| root | 1.61 |

The samples were inoculated with yeasts from a saturated culture in YPD medium. 35 μl of culture from each strain, White Yeast 1, Slow Growth Yeast 2 and Yellow Yeast 3, were inoculated. These media were agitated at 240 rpm and 28° C. for 48 hours; next cellular growth and amount of sugar in the samples was measured to determine sugar consumed.

| Sample | Sugar percentage (%) | OD₆₀₀ |
|---|---|---|
| Green stalk | 1.710 | 1.221 |
| Woody stalk | 0.93 | 1.411 |
| Leaf | 1.98 | 1.363 |
| Root | 0.77 | 0.865 |

Data show how yeasts consumed initial sugar. Agitation of media stopped to impede oxygenation thus allowing beginning of fermentation. As well as in previous figures, bubbles can be seen in FIGS. 23 and 24 as product of fermentation.

The experiment was repeated in media wherein sugar concentrations were higher. To increase content of sugars in media, an acid hydrolysis of vegetal matter was carried out thus obtaining the following sugar concentrations:

| | initial sugar |
|---|---|
| sample | percentage (%) |
| green stalk | 3.850 |
| woody stalk | 16.59 |
| leaf | 8.63 |
| root | 18.50 |

At this time strains from YPD medium saturated culture were inoculated with 35 μl of culture from each strain. These media were kept under agitation at 240 rpm and 28° C. for 48 hours measuring cellular growth once this time elapsed.

| sample | OD₆₀₀ |
|---|---|
| green stalk | 1.670 |
| woody stalk | 1.183 |
| leaf | 2.078 |
| root | 1.881 |

Therefore it can be confirmed that increasing sugar content does not affect yeasts.

Discussion

Different factors can be deduced from observed results previously presented. The simultaneous use of the three yeast strains of the present application in the fermentation process, that is in a coordinated manner can confer greater quality whatever the fermentation product to be obtained may be, since the coordinated action of the three yeasts will enhance in itself the fermentation process. As these three yeasts were obtained from casks, the three must be at the end of the fermentation process wherein ethanol concentrations approach 16-19 degrees. Better functioning yeast in glucose (Yellow Yeast 3) can cover the other two in media of high content of same (they must survive in 70% sucrose anyhow, confirmed by results obtained), Slow Growth Yeast 2 can create a veil favoring oxygen absence providing all the necessary advantages to produce a better fermentation and lastly White Yeast 1 can grow in a medium with very high ethanol concentrations, maybe protecting the other yeast species present in the process, so in this manner—protecting the set of yeasts—a further enhancement of obtained juices is achieved or whatever the obtained product may be. Consequently, in any process wherein fermentation is present, from fermentable sugars, these yeasts can presumably provide a higher quality to the product obtained. Therefore this would explain why a higher quality of the product obtained through different fermentable sources would be achieved (sucrose, molasses, starch, must, etc.).

On the other hand this coordinated action together with each strain's own characteristics (analyzed in the present Description) explains why solutions with a very elevated sucrose concentration (or succedaneums) can ferment and thus obtain products with a very high ethanol concentration (most yeast strains cannot endure ethanol concentrations over 13%.)

Coordinated action of these three yeasts together with initial presence of greater amounts of sugars makes the fermentation richer in secondary products. Diverse matters, acting as fermentation sources and covering a wide spectrum of representative products of the fermentation industry as sugar, starch, molasses and the rest of vegetables, etc. have been analyzed. Microorganisms need adequate conditions to grow, reproduce and develop. This is not always possible. Since the three yeasts studied in the present application are capable of fermenting in a simple medium containing starch, it can be inferred that they might be useful in those processes using starch as previous origin to the carbon source for fermentation. It is known that yeasts are capable of fermenting hydrolyzed starch and that yeasts are nevertheless necessary in the production of beer.

Thus it might be inferred from previous analysis herewith stated that from mixture of these yeasts the following can be obtained:

1.—greater ethanol concentrations

2.—higher quality products

3.—fermentation of non fermenting matters to date due to their high sugar content and ensuing high ethanol production.

Outlining Positive Results
Obtainment of higher alcohol graduation beverages
Obtainment of alcohol with a greater energetic efficiency since a higher ethanol concentration implies less amount of energy invested in distillation.
Obtainment of higher quality wine, bread and beer since yeasts are needed to obtain all of these foodstuffs, attainment of such through yeasts described in the present application will produce a higher quality product.
A more effective bio ethanol elaboration can be achieved from vegetal or non vegetal biomass consequently production costs can be lower than present ones. This means a substantial improvement regarding bio ethanol obtainment by conventional processes from any of its origins as to carbon source, since whatever said source might be fermentation substrate is glucose, to be later on turned into ethanol by yeasts of the Saccharomyces cerevisiae (mainly) species. Industries handling higher volume and thus receiving higher benefits will be among others, those involved in production of bio ethanol as fuel, and also alcohol production industries, producing alcohol from low sucrose or glucose concentration sources being poorly cost efficient.
Obtained results point out to the fact that yeasts studied in the present application are generally able to improve energetically and economically, any process based on fermentation of sugars. Therefore the use of herewith described yeasts shall not be discarded, virtually including production of any alcoholic beverage obtained from plants or derivatives as fruits, from their sugars already extracted or extracting them.

Work of Yeasts Presented in the Present Application:
Yeasts work coordinately, there is no other way to explain each of them having an outstanding role, that is a synergy phenomenon is present.
Yellow yeast 3 could be used in those processes needing a yeast with great resistance to osmotic stress, furthermore capable of growing, developing, reproducing and fermenting, osmotic stress is a problem of struggle for existing water in an aqueous medium; a very high glucose concentration implies a defensive system notably effective to avoid harm from strong pressure, more glucose in the medium means more external pressure in the cell towards the exterior. Thus it is a phenomenon having many physical, chemical and biological implications. This phenomenon is of such importance that many research groups keep studying it worldwide for many years to learn about damages and responses in the cells. Yeast S. cerevisiae has been specially used for this purpose. Presently there are many research groups focused on enhancing some of the crucial steps, including damage and resistance, to obtain at the end yeasts as Yellow Yeast 3, aggravated by the fact that those would be genetically modified and the yeast of the present application is a yeast obtained through natural selection.
White Yeast 1 has a very high ethanol tolerance; talking about an ethanol concentration of 60% in plate and 70% v/v in liquid, means talking about non reported numbers in the art to date in any scientific publication or patent application for S. cerevisiae strains. As in the case of glucose resistance, described values multiply those observed to date in different magnitudes. This means that strains of the present application: White Yeast 1, Slow Growth Yeast 2 and Yellow Yeast 3, possess a potential to solve scientific and technical problems of magnitudes superior to those of known strains to date. In case of White Yeast 1 the role it might potentially develop should be stressed. Ethanol is toxic for the majority of live organisms, its toxicity depending on the species and concentrations. Ethanol has been used since ancient times to disinfect all types of materials in medical environment, normally the majority of pathogen bacteria species are affected by ethanol, causing their death. White Yeast 1 might have applications in this scope, it can be used to study how microorganisms survive (specially a model organism as S. cerevisiae yeast) at elevated ethanol concentrations and thus determine the keys to the process. It can also serve to transport microorganisms non resistant to elevated concentrations of ethanol for that type of solutions. An elevated concentration of White Yeast 1 might create a micro environment allowing survival of certain microorganisms during a short period of time. The same could be stated for Yellow Yeast 3 for elevated sugar concentrations. This type of strategy is used when for example techniques of electroporation of DNA in protoplasts are carried out, avoiding action of exo and endonuclease (protecting DNA) by using elevated concentrations of a DNA different from the object of electrophoration as for example, salmon sperm.
Yeasts are oxidative, fermentative or its metabolic activity is of both types at the same time. In the surface of a liquid, oxidative yeasts can grow in form of film, veil or foam, that is the reason why they are called film forming yeasts. Slow Growth Yeast 2 is especially sensitive in its morphologic plasticity to changes produced by key fermentation aspects. For example, when its development in YPD plate is observed (see FIGS. 3 and 4), a quick blackening is appreciated as time goes by. If a fast change is observed in said yeasts (it shifts rapidly from an intense white color to a strong black one; yeasts in their normal state have a white color), the reason might be they are in contact with a factor not regularly experienced, this factor is probably oxygen. This thesis is supported by the fact that blackening is first observed and in a more intense manner in the part of the yeast in contact with oxygen in its highest concentration (surface of the YPD medium). This hypothesis is reinforced by observation that this phenomenon happens when Slow Growth Yeast 2 is in the form of mycelium and not in spherical form. This argument is coherent with the fact that fermentation is a partial combustion process of glucose in absence of oxygen. Thus it will be more effective as less oxygen is present. In this manner it might be inferred that Slow Growth Yeast 2 could be contributing to the fermentation process happening in better conditions, guaranteeing a veil protecting it from the presence of oxygen. Consequently, an immediate commercial use could be achieved by integrating it to fermentation processes whatever their typology, to produce a better process, more effective and of higher quality. In general, sugars are the most appropriate energetic source for yeasts, although oxidative ones for example, those forming films, oxide organic acids and alcohol and also contribute in the production of the flavors or “bouquet” of wines. Thus a commercial use of this yeast could imprint a bouquet on wine strains, or any other fermentation process as production of liquors, low grade alcoholic beverages, bread, etc. It could also be used in any industrial process of those previously mentioned or wherever the absence of oxygen might be specially necessary or important, allowing the use of a film forming yeast, as for example in the production of a drug or substance sensitive to oxygen. This group could encompass all substances considered as antioxidants, that is, those especially sensitive to electronic unbalance in their environment, such electronic unbalance characterized by an excess or defect of electronic charge in relation to a basal state.
This same yeast could be used to protect specific solutions from oxidation, as a method to avoid loss of natural properties in foodstuffs, or industrial solutions, sensitive to oxygen, (produced or not by oxygen), meaning the presence of an agent generating electronic unbalance, capturing electrons (for example the case of oxygen) or ceding electrons (for example the case of Fe2+). This type of known substances known as antioxidants includes a large group of substances of biomedical interest and at the same time beneficial to a great majority of living organisms since its main function is acting as tampons of an electronic unbalance. For example, those substances of interest in aging processes and cancer, as antioxidants: citric acid, ascorbic (vitamin C), glutathione, resveratrol, etc. In general, they are substances acting as pH indicators act that is, easily changing their electronic conformation, gaining or loosing electrons before relatively small changes in the electronic balance of the environment they are in. Manufacture, transportation or storage of this type of substance creating a veil of this type, would be clearly enhanced. Afterwards, this veil could be collected in a simple centrifugation process. This could be for example of great interest for biomedicine or in alimentary sectors where the use of antioxidant substances added to foodstuffs has been of increasing interest in these last years. Also in industrial sectors producing very sensitive substances to oxidation. Presently this place of the art could be occupied by other techniques as vacuum manufacturing or packing, but in this case, the use of this type of yeasts could mean a great cost reduction and in some cases an enhancement in product quality, since vacuum implementation could suppose some impact in the product at the moment of production.

Once the invention has been sufficiently described, as well as some preferred embodiments of same, it should only be added that modifications to its constitution and materials used are possible without departing from its scope, defined in the ensuing claims.

1. A culture method of a vineyard resistant to meteorological and/or climatic roughness through development of deep and strong roots, characterized by
selection of autochthonous natural grapevine varieties, having a natural wild grapes' origin with no grafting whatsoever,
fertilizing said grapevines with compost obtained from the soil's own herbs,
not adding other nutrients but those of the own soil,
collecting grapes after autumn rainfall

2. A thorough fermentation method with wild grape yeasts reminiscing the soil and possessing its organoleptic properties characterized in that the totality of the sugars contained in the grapes ferment through:
formation of a wild grapes' juice barrel in deposits of a capacity inferior to 5,000 liters over 20-25% of said volume,
reproduction of obtained autochthonous wild yeasts, reaching 3.5 to 6% volume of alcohol,
posterior topping ups every 7 to 15 days,
fermentation with the same yeasts stepwise until fermentation of all the sugar obtaining a final alcoholic grade wine between 14 to 20% volume.

3. A method to obtain wild grape yeasts according to claim No. 2 characterized by obtaining three different yeasts: White yeast (1), Slow Growth Yeast (2) and Yellow Yeast (3), wherein the extract of the mixture obtained from said three yeasts is obtained in YPD medium and by separation of said yeasts through culture in YPD medium with different concentrations of alcohol and sugar:
White Yeast (1) through growth in YPD media containing ethanol concentrations up to 60%.
Slow Growth Yeast (2) through growth in YPD media containing ethanol concentrations up to 25%.
Yellow Yeast (3) through growth in YPD media containing sugar, such that, in said medium, it produces colonies of bigger size than the rest of detected yeasts.

4. A method for use of individual wild yeasts according to claim No. 3 characterized in that individual extract of said three yeasts finds application in:
White Yeast (1) surviving in high ethanol concentrations
Slow Growth Yeast (2) forming a mycelium film beneficial to fermentation process.
Yellow Yeast (2) facilitating yeast growth in glucose concentrations.

5. A method for use in industrial production of alcohols from wild yeasts according to claim No. 3 characterized in that the mixture obtained in said three yeasts, White Yeast (1), Slow Growth Yeast (2) and Yellow Yeast (3) ferments in low as well as in high concentrations of sugars present in solutions of sucrose; commercial sugar; sugar cane molasses and beet molasses; sugars from starch hydrolysis and from vegetal matters, and others.

6. A method for use in the production industry of low grade alcoholic beverages from wild yeasts according to claim No. 3 characterized in that the mixture obtained from said three yeasts, White Yeast (1), Slow Growth Yeast (2) and Yellow Yeast (3) allows through fermentation of sugars from different sources, obtainment of low grade alcoholic beverages as wine, beer, cider, etc.

7. A method for use in the production industry of high grade alcoholic beverages from wild yeasts according to claim No. 3 characterized in that the mixture obtained from said three yeasts, White Yeast (1), Slow Growth Yeast (2) and Yellow Yeast (3), allows through fermentation of sugars from different sources, obtainment of high grade alcoholic beverages as cognacs, rums, vodkas, etc.

8. A method for use of wild yeasts in the bakery industry, according to claim No. 3 characterized in that the starch fermentation process uses said mixture obtained from said three yeasts, White Yeast (1), Slow Growth Yeast (2) and Yellow Yeast (3).
